(19) United States
(12) Patent Application Publication
(10) Pub. No.: US 2016/0310399 A1
(43) Pub. Date: Oct. 27, 2016

(54) SUNLESS TANNING

(71) Applicant: CONOPCO, INC., d/b/a UNILEVER, Englewood Cliffs, NJ (US)

(72) Inventors: Ranjit Kaur BHOGAL, Huntingdon (GB); John CASEY, Yelden (GB); Eleanor Margaret D'AGOSTINO, Rushden (GB)

(73) Assignee: Conopco, Inc., d/b/a UNILEVER, Englewood Cliffs, NJ (US)

(21) Appl. No.: 15/102,608

(22) PCT Filed: Nov. 24, 2014
(86) PCT No.: PCT/EP2014/075394

§ 371 Date: Jun. 8, 2016

(30) Foreign Application Priority Data
Dec. 20, 2013 (EP) 13198722.4

(51) Int. Cl. A61K 8/66; A61Q 17/04; A61K 8/22; A61K 8/42; A61Q 19/04; A61K 8/49

(52) U.S. Cl. CPC A61K 8/66; A61Q 19/04; A61K 8/498; A61K 8/22; A61K 8/42; A61Q 17/04

(57) ABSTRACT

This invention relates to a topical sunless tanning composition, in particular a topical sunless tanning composition which provides an immediate colour effect. There is much interest in effectuating a tan via cosmetic means. Most prominent among the sunless tanning agents is dihydroxyacetone (DHA, which is also chemically known as 1,3-dihydroxy-2-propanone). However, a problem with using DHA to tan the skin is the length of time it takes for the skin to develop colour (from 3 to 12 hours after application in general). Thus, in a first aspect of the invention, a topical sunless tanning composition is provided, the topical sunless tanning composition comprising: (a) 0.01-10, preferably 0.1-5 w/w a polyphenol selected from the group consisting of (+)-catechin, (−)-catechin, (+)-epicatechin, (−)-epicatechin, epigallocatechin gallate, (−)-epigallocatechin, (−)-catechin gallate, epicatechin gallate, or mixtures thereof; (b) A peroxidase or laccase (c) 0.0001-3 preferably 0.001-1, most preferably 0.01-1 w/w hydrogen peroxide or a hydrogen peroxide generator when peroxidase is present; and (e) A dermatologically acceptable vehicle.

This invention relates to a topical sunless tanning composition, in particular a topical sunless tanning composition which provides an immediate colour effect.

Today there is a great health concern with natural tanning through sunlight. Ultraviolet radiation from the sun is considered to be a leading factor in causing skin cancer. Even if not lethal, ultraviolet radiation has been acknowledged at accelerating ageing and wrinkling processes on the skin.

Beyond health concerns, there are obvious practical reasons against natural tanning. Foremost is the reason that in many areas of the globe and during all but summer time, there is insufficient sunlight available to accomplish a natural tan.

Based on the above considerations, there is much interest in effectuating a tan via cosmetic means.

Most prominent among the sunless tanning agents is dihydroxyacetone (DHA, which is also chemically known as 1,3-dihydroxy-2-propanone). DHA, after application, is believed to exert its effect through interactions between, for example, its keto group and the amino groups of amino acids and peptides naturally occurring in the stratum corneum of the skin. These so-called Maillard reactions are believed to lead to formation of brown pigments in the skin, thereby giving it an appearance similar to that of a naturally obtained tan.

However, a problem with using DHA to tan the skin is the length of time it takes for the skin to develop colour (from 3 to 12 hours after application in general). The rate of colour development is often too slow for many consumers seeking an “instant” benefit. It would therefore be advantageous to develop a sunless tanning product that colours the skin more quickly or immediately on application. In addition, because colour development is not immediate, it is also often difficult to see where a DHA containing formulation has been applied and where it has not. This can result in uneven application, leading to a streaky tan.

U.S. Pat. No. 6,399,046 (Beiersdorf) discloses the use of catechins or gallic esters of catechins or aqueous or organic extracts from plants or parts of plants which have a content of catechins or gallic esters of catechins, for example the leaves of the plant family Theaceae, in particular of the species Camellia sinensis (green tea) or a typical ingredient thereof (such as, e.g. polyphenols or catechins, caffeine, vitamins, sugars, minerals, amino acids, lipids), for intensifying natural skin tanning or for stimulating melanogenesis in human skin.

WO 2009/082735 (University of Chicago and Loyola University Chicago) discloses an enzymatic and a chemical synthesis of melanins and novel melanins. The synthesized melanins inhibit the binding of viruses to animal cells, prevent a virus from infecting the cells of its host, and prevent the spread of viral infections from person-to-person. The method comprises the step of contacting a polyphenol oxidase (PPO) and a pyrocatechol substrate for a time sufficient to produce a melanin. In one embodiment, the pyrocatechol substrate is a bicyclic compound such as esculetin, dapthnetin, catechin, baicalein or alizarin. The compounds of the invention may be administered topically to the skin or mucosa, either dermally or transdermally.

US 2003/0103917 (Pruche) discloses a dyeing composition promoting natural skin pigmentation. The composition comprises, in a physiologically acceptable medium, at least one enzyme having a pro-pigmenting activity and an efficient amount of a catalytic system comprising a first component selected amongst salts and oxides of Mn(II) and/or Zn(II) and the mixtures thereof and a second component selected amongst alkaline hydrogenocarbonates, alkaline earth hydrogenocarbonates and the mixtures thereof, the proportions of the first component and the second component being defined. The enzymes can be selected, for example, amongst pyranose oxidases, glucose oxidases, glycerol oxydases, lactate oxydases, pyruvate oxydases, uricases, choline oxydases, sarcosine oxydases, bilirubin oxydases, laccases, tyrosinases, peroxydases, catalases, superoxydesdimutases and the mixtures thereof, or amongst plant and animal extracts containing the above-mentioned enzymes. The composition additionally comprises an efficient amount of at least one oxidation colouring agent precursor selected amongst compounds having at least one aromatic cycle with at least two hydroxyl groups (OH) carried by two consecutive carbon atoms of the aromatic cycle. The preferred colouring agent precursors are, amongst others, flavanols such as catechin and epicatechin gallate. The composition can also contain an efficient amount of at least one amino acid comprising at least one thiol group (SH). The preferred amino acids include cysteine and the derivates thereof, more particularly L-cysteine and L-cysteins hydrochloride, gluthatione and the derivates thereof.

### SUMMARY OF THE INVENTION

The inventors have observed that when catechins and green tea extract are used in combination with peroxidase or laccase, the colour produced on artificial skin is significantly enhanced compared to using catechins and green tea extract alone. In addition, the inventors have observed that when (+)-catechin and peroxidase enzyme were used in combination with DHA and amino acids to tan artificial skin, colour production was more immediate (compared to colour production after treatment with DHA and amino acid alone). The inventors also observed that over longer time periods, colour production was enhanced when (+)-catechin and peroxidase enzyme were used in combination with DHA and arginine or DHA and cysteine.

Thus, in a first aspect of the invention, a topical sunless tanning composition is provided, the topical sunless tanning composition comprising:
(a) 0.01-10, preferably 0.1-5% w/w a polyphenol selected from the group consisting of (+)-catechin, (−)-catechin, (+)-epicatechin, (−)-epicatechin, epigallocatechin gallate, (−)-epigallocatechin, (−)-catechin gallate, (−)-epicatechin gallate, or mixtures thereof;
(b) A peroxidase or laccase
(c) 0.0001-3 preferably 0.001-1, most preferably 0.01-1% w/w hydrogen peroxide or a hydrogen peroxide generator when peroxidase is present; and
(e) A dermatologically acceptable vehicle.

A CIE 1976 L*a*b* (CIELAB) ΔE value of less than 5 using VeriVide DigiEye v2.6 software was considered not to tan skin.

In a second aspect of the invention, a method of sunless tanning the skin is provided, the method of sunless tanning the skin comprising the step of applying to the skin the topical sunless tanning composition of the first aspect of the invention.

### DETAILED DESCRIPTION OF THE INVENTION

In a first aspect of the invention, a topical sunless tanning composition is provided, the topical sunless tanning composition comprising:
(a) 0.01-10, preferably 0.1-5% w/w a polyphenol selected from the group consisting of (+)-catechin, (−)-catechin, (+)-epicatechin, (−)-epicatechin, epigallocatechin gallate, (−)-epigallocatechin, (−)-catechin gallate, (−)-epicatechin gallate, or mixtures thereof;
(b) A peroxidase or laccase
(c) 0.0001-3 preferably 0.001-1, most preferably 0.01-1% w/w hydrogen peroxide or a hydrogen peroxide generator when peroxidase is present; and
(e) A dermatologically acceptable vehicle.

The polyphenol is preferably selected from the group consisting of (+)-catechin or (−)-epicatechin and (−)-epigallocatechin gallate.

The peroxidase is preferably a non-animal haem peroxidase from class II (fungi) or class III (plants and algae). The peroxidase is preferably obtained from the group consisting of Arabidopsis thaliana, horse radish, barley, peanut soy bean, tobacco, and turnip (plants), Chlorophyta spirogyra (green algae), Arthromyces ramosus and Corprinus cinereus (fungi), most preferably the peroxidase is horse radish peroxidase or soy bean peroxidase.

The laccase is preferably selected from the group consisting of cyanobacteria of the genuses Leptolyngbya, Oscillatoria and Phormidium; bacteria of the genuses Bacillus, Escherichia, Pseudomonas, Shigella, Sinorhizobium, Stenotrophomonas, Streptomyces, and Thermus; fungi of the genuses Agaricus, Agrocybe, Albatrellus, Athelia, Botryotinia, Cantharellus, Ceriporiopsis, Cerrena, Chaetomium, Cladosporium, Clitocybe, Coniothyrium, Coprinopsis, Coriolisimus, Coriolopsis, Cortinarius, Cryptococcus, Cyathus, Daedalea, Emericella, Fomes, Fomitella, Fusarium, Ganoderma, Hypocrea, Inocybe, Lactarius, Lentinula, Lepiota, Lepista, Leptonia, Loweporus, Lyophyllum, Magnaporthe, Marasmius, Melanocarpus, Myceliophthora, Myriogonium, Myrothecium, Neurospora, Panus, Paraconiothyrium, Parasola, Peltigera, Penicillium, Peniophora, Perenniporia, Phellinus, Phlebia, Phoma, Pleurotus, Podospora, Polyporus, Pycnoporus, Ramaria, Rhizoctonia, Rigidoporus, Russula, Scytalidium, Solorina, Steccherinum, Trametes, Tricholoma, Trichophyton, Volvariella; plants of the genuses Acer, Mangifera, Pistacia, Pleiogynium, Populus, Prunus, Rhus, Schinus and Toxicodendron; and mixtures thereof.

The hydrogen peroxide generator typically comprises a hydrogen peroxide generating oxidase, a substrate and oxygen. The hydrogen peroxide generating oxidase is preferably selected from the group consisting of (S)-2-hydroxy acid oxidase, D-galactose oxidase, glucose oxidase, coniferyl alcohol oxidase, glycolate oxidase, hexose oxidase, oxalate oxidase, amino acid oxidase and L-galactonolactone oxidase and the respective substrate is selected from the group consisting of (S)-2-hydroxy acid, D-galactose, glucose, coniferyl alcohol, a-hydroxy acids, D-glucose, oxalic acid, amino acid and L-galactono-1,4-lactone. Thus the hydrogen peroxide generator is preferably selected from the group consisting of (S)-2-hydroxy acid with (S)-2-hydroxy acid oxidase, D-galactose with D-galactose oxidase, glucose with glucose oxidase, coniferyl alcohol with coniferyl alcohol oxidase, α-hydroxy acids with glycolate oxidase, D-glucose with hexose oxidase, oxalic acid with oxalate oxidase, and L-galactono-1,4-lactone with L-galactonolactone oxidase, amino acid oxidase with amino acids, all in the presence of oxygen.

In one embodiment, the topical sunless tanning composition further comprises 0.01-25, preferably 0.1-15, most preferably 0.1-10% w/w 1,3-dihydroxyacetone dimer and/or D-erythrulose.

In one embodiment, the topical sunless tanning composition further comprises 0.01-10, preferably 0.1-5% w/w an amino acid, preferably an amino acid excluding a thiol group.

Preferably the amino acid is selected from the group consisting of glycine, L-lysine, L-arginine, L-cysteine and mixtures thereof, more preferably selected from the group consisting of glycine, L-lysine, L-arginine and mixtures thereof.

In another embodiment, the topical sunless tanning composition further comprises 0.01 to 15, preferably 0.1 to 10, most preferably 0.5 to 7.5% w/w an inorganic sunscreen and/or organic sunscreen.

Sunscreens include those materials commonly employed to block ultraviolet light. Illustrative organic compounds are the derivatives of p-aminobenzoic acid (PABA), cinnamate and salicylate. For example, avobenzophenone (Parsol 1789®), octyl methoxycinnamate and 2-hydroxy-4-methoxy benzophenone (also known as oxybenzone) can be used. Octyl methoxycinnamate and 2-hydroxy-4-methoxy benzophenone are commercially available under the trade marks, Parsol MCX and Benzophenone-3, respectively. Ecamsule, a benzylidene camphor derivative, sold under the trade mark Mexoryl SX, and drometrizole trisiloxane, a benzotriazole sold under the trade mark Mexoryl XL, may also be used. Still other examples include octocrylene, phenylbenzimidazole sulfonic acid (also known as ensulizole), ethylhexyl salicylate,diethylhexyl naphthylate, bimotrizinole (trade marked as Tinosorb S) and bisoctrizole (Tinosorb M).

Inorganic sunscreens include oxides like titanium dioxide and zinc oxide which reflect or scatter the sun's rays. The exact amount of sunscreen employed in the topical sunless tanning composition can vary depending upon the degree of protection desired from the sun's UV radiation.

The dermatologically acceptable carrier may be aqueous-based, anhydrous or an emulsion whereby a water-in-oil or oil-in-water emulsion is generally preferred. If the use of water is desired, water typically makes up the balance of the topical sunless tanning composition, and preferably makes up from 5 to 98%, and most preferably from 40 to 80% by weight of the topical sunless tanning composition, including all ranges subsumed therein.

In addition to water, organic solvents may be optionally included. Illustrative and non-limiting examples of the types of organic solvents suitable for use in the present invention include alkanols like ethyl and isopropyl alcohol, mixtures thereof or the like.

Other suitable organic solvents include ester oils like isopropyl myristate, cetyl myristate, 2-octyldodecyl myristate, avocado oil, almond oil, olive oil, neopentylglycol dicaprate, mixtures thereof or the like. Typically, such ester oils assist in emulsification, and an effective amount is often used to yield a stable, and most preferably, water-in-oil emulsion.

Emollients may also be used, if desired. Alcohols like 1-hexadecanol (i.e. cetyl alcohol) are often desired as are the emollients generally classified as silicone oils and synthetic esters. Silicone oils suitable for use include cyclic or linear polydimethylsiloxanes containing from 3 to 9, preferably from 4 to 5, silicon atoms. Non-volatile silicone oils useful as an emollient material in the inventive composition described herein include polyalkyl siloxanes, polyalkylaryl siloxanes and polyether siloxane copolymers. The essentially non-volatile polyalkyl siloxanes useful herein include, for example, polydimethylsiloxanes. Silicone elastomers may also be used.

The ester emollients that may optionally be used are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, stearyl stearate and arachidyl behenate.
(5) Sterols esters, of which cholesterol fatty acid esters are examples.

Emollients, when used, typically make up from 0.1 to 50% by weight of the topical sunless tanning composition, including all ranges subsumed therein.

Fatty acids having from 10 to 30 carbon atoms may also be included as acceptable carriers within the topical sunless tanning composition of the present invention. Illustrative examples of such fatty acids include pelargonic, lauric, myristic, palmitic, stearic, isostearic, oleic, linoleic, arachidic, behenic or erucic acid, and mixtures thereof. Compounds that are believed to enhance skin penetration, like dimethyl sulfoxide, fatty acids and ethanol may also be used as an optional carrier.

Humectants of the polyhydric alcohol type may also be employed in the topical sunless tanning compositions. The humectant often aids in increasing the effectiveness of the emollient, reduces scaling at the skin surface, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. Other humectants which may be used include hydroxyethyl urea. The amount of humectant may range anywhere from 0.2 to 25%, and preferably, from 0.5 to 15% by weight of the topical sunless tanning composition, including all ranges subsumed therein.

Moisturisation may be improved through use of petrolatum or paraffins.

Thickeners may also be utilized as part of the dermatologically acceptable carrier in the topical sunless tanning compositions. Typical thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0 to 5, usually from 0.001 to 1, optimally from 0.01 to 0.5% by weight of the topical sunless tanning composition, including all ranges subsumed therein.

Collectively the water, solvents, silicones, esters oils, emollients, fatty acids, humectants and/or thickeners will constitute the dermatologically acceptable carrier in amounts from 1 to 99.9, preferably from 80 to 99% by weight of the topical sunless tanning composition.

Surfactants may also be present in the topical sunless tanning composition of the invention. Total concentration of the surfactant will range from about 0 to about 40, and preferably from about 0 to about 20, optimally from about 0 to about 5% by weight of the topical skin lightening composition. The surfactant may be selected from the group consisting of anionic, nonionic, cationic and amphoteric actives. Particularly preferred nonionic surfactants are those with a C10-C20 fatty alcohol or acid hydrophobe condensed with from 2 to 100 moles of ethylene oxide or propylene oxide per mole of hydrophobe; mono- and di-fatty acid esters of ethylene glycol; fatty acid monoglyceride; sorbitan, mono- and di-C8-C20 fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C8-C20 acyl isethionates, acyl glutamates, C8-C20 alkyl ether phosphates and combinations thereof.

Fragrances may be used in the topical sunless tanning composition. Illustrative non-limiting examples of the types of fragrances that may be used include those comprising terpenes and terpene derivatives like those described in Bauer, K., et al., Common Fragrance and Flavor Materials, VCH Publishers (1990). Illustrative yet non-limiting examples of the types of fragrances that may be used in this invention include myrcene, dihydromyrenol, citral, tagetone, cis-geranic acid, citronellic acid, mixtures thereof or the like. Preferably, the amount of fragrance employed in the topical skin lightening composition is in the range from 0.0 to 10, more preferably 0.00001 to 5, most preferably 0.0001 to 2% by weight of the topical sunless tanning composition, including all ranges subsumed therein.

Various types of optional additional active ingredients may be used in the topical sunless tanning compositions. Actives are defined as skin benefit agents other than emollients and other than ingredients that merely improve the physical characteristics of the composition. Although not limited to this category, general examples include extender pigments such as talcs and silicas, as well as alpha-hydroxy acids, beta-hydroxy acids and zinc salts.

Beta-hydroxy acids include salicylic acid, for example. Zinc oxide and zinc pyrithione are examples of zinc salts useful in the topical skin lightening composition.

Many compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives are methyl paraben, propyl paraben, phenoxyethanol and benzyl alcohol. Preservatives will usually be employed in amounts ranging from 0.1 to 2% by weight of the topical sunless tanning composition.

Still other optional ingredients that may be used with the topical sunless tanning composition include dioic acids (e.g. malonic acid and sebacic acid), antioxidants like vitamin E, retinoids, including retinoic acid, retinal, retinol and retinyl esters such as retinyl propionate and retinyl palmitate, conjugated linoleic acid, petroselinic acid and mixtures thereof, as well as any other conventional ingredients well known for wrinkle-reducing (such as hyaluronic acid, ubiquinone, jasmonic acid derivatives, collagen, peptides and proxylane), anti-acne effects and reducing the impact of sebum.

When making the topical sunless tanning composition, the desired ingredients are mixed in no particular order and usually at temperatures from 70 to 80° C. and under atmospheric pressure.

The packaging for the topical sunless tanning composition can be a bottle, roll-ball applicator, propellant driven aerosol device, squeeze container. For a topical sunless tanning composition comprising laccase, the composition will need to be in packaged under an oxygen free atmosphere. For a topical sunless tanning composition comprising peroxidase, the composition will need to separate the peroxidase and hydrogen peroxide components, or where an oxidoreductase based hydrogen peroxide generator is used, package the topical sunless tanning composition in an oxygen free environment.

In a second aspect of the invention, a method of sunless tanning the skin is provided, the method of sunless tanning the skin comprising the step of applying to the skin the topical sunless tanning composition of the first aspect of the invention.

### EXAMPLES

### Example 1

### A Comparison of the Colour Development on Artificial Skin Following Treatment with (+)-catechin, or (+)-catechin and Horseradish Peroxidase

Materials

Vitro-Skin, IMS Inc., USA

Hydrogen peroxide, Sigma, UK

(+)-catechin, Sigma, UK

Dimethyl sulphoxide (DMSO), Sigma, UK

Horseradish peroxidase type VI, Sigma, UK (274 U/mg (1 Unit (U)=1 mg purpurogallin in 20 seconds at 20 degrees centigrade at pH 6)

Sodium citrate buffer pH 5.5

Method

Using a pencil, circles of 2.5 cm diameter were marked out on sheets of Vitro-Skin. The Vitro-Skin was then hydrated overnight by placing in a humidifying chamber at room temperature at 50% RH. The next morning the colour of the area within each circle was measured by recording CIE 1976 L*a*b* (CIELAB) values using VeriVide DigiEye v2.6 software. L*, a* and b* values describe a colour. The L* value (lightness) ranges from 0, which represents black, to 100, which represents white. The a* value relates to the redness/greeness, with +a* denoting red and −a* denoting green. The b* value relates to yellowness/blueness, with +b* denoting yellow and −b* denoting blue.

Two samples were prepared as follows:
1. (+)-Catechin: 800 μL sodium citrate buffer (100 mM pH 5.5; final concentration 80 mM), 100 μL milliQ water and 100 μL catechin (10 mg/mL stock in DMSO; final concentration 1 mg/mL) were combined in a plastic Bijou pot and mixed gently.
2. (+)-Catechin/HRP: 700 μL sodium citrate buffer (100 mM pH 5.5), 100 μL 3% hydrogen peroxide (final concentration 0.3%), 100 μL catechin (10 mg/mL stock in DMSO; final concentration 1 mg/mL) and 100 μL horseradish peroxidase (100 units/mL in citrate buffer pH 5.5) were combined in a plastic Bijou pot and mixed gently. The final concentration of sodium citrate buffer was 80 mM. 30 μL of each sample were then applied to the Vitro-Skin circles and rubbed in for 10 seconds using a gloved fingertip. Each sample was tested in triplicate. The Vitro-Skin was then incubated at 35° C. and 50% RH. CIE 1976 L*a*b* measurements for each sample area were then recorded at various time-points up to 5 days. The magnitude of colour change (ΔE) was calculated using the following equation:

ΔE=\sqrt{(L*_{S}−L*_{B})²+(a*_{S}−a*_{B})²+(b*_{S}−b*_{B})²}

where B=blank (unstained) and S=stained. Statistical analyses were performed using Student's t test.

Results

The results are set forth in Table la from which it is apparent that the colour change of (+)-catechin/horseradish peroxidase treated Vitro-Skin was significantly greater than the colour change of (+)-catechin treated Vitro-skin at all time points (p=<0.0001).

TABLE 1a
ΔE values for Vitro-Skin treated with (+)-catechin +/− horseradish
peroxidase (HRP). Standard deviations at 95% confidence limits are
provided.

| | | ΔE | p value |
|---|---|---|---|
| | ΔE (+)-catechin | (+)-catechin/HRP | (ΔE (+)-catechin vs ΔE (+)- |
| | treated | treated | catechin/HRP) |
| 1 h | 0.74 +/− 0.71 | 12.18 +/− 0.28 | <0.0001 |
| 18 h | 0.87 +/− 0.37 | 9.45 +/− 0.34 | <0.0001 |
| 24 h | 1.28 +/− 0.43 | 9.82 +/− 0.34 | <0.0001 |
| 42 h | 0.63 +/− 0.27 | 10.16 +/− 0.13 | <0.0001 |
| 48 h | 0.95 +/− 0.53 | 9.91 +/− 0.24 | <0.0001 |
| 98 h | 1.33 +/− 0.35 | 9.93 +/− 0.07 | <0.0001 |

Table 1b shows the CIE 1976 L*a*b* (CIELAB) values at the 98 hour time-point from which it is clear that a noticeable browner colour was achieved after treatment with (+)-catechin/horseradish peroxidase compared to treatment with (+)-catechin alone.

TABLE 1b
L*a*b* values for untreated Vitro-Skin and Vitro-Skin treated with
(+)-catechin +/− horseradish peroxidase for 98 hours. Standard deviations
at 95% confidence limits are provided.

| | | | (+)-catechin/HRP |
|---|---|---|---|
| | Untreated | (+)-catechin treated | treated |
| L* | 89.36 +/− 0.64 | 88.31 +/− 0.10 | 90.64 +/− 0.43 |
| a* | 1.13 +/− 0.16 | 0.84 +/− 0.14 | 1.49 +/− 0.09 |
| b* | −7.02 +/− 0.09 | −7.06 +/− 0.08 | 2.77 +/− 0.30 |

Conclusions

These results demonstrate that, on artificial skin, a significantly greater colour development is obtained after treatment with (+)-catechin and horseradish peroxidase, compared to the colour development obtained after treatment with (+)-catechin alone.

### Example 2

### A Comparison of the Colour Development on Artificial Skin Following Treatment with (+)-catechin, or (+)-catechin and Laccase

Materials (Additional)

Laccase 51003, Novozymes, Denmark

Method

Vitro-Skin was prepared as described in Example 1.

Two samples were prepared as follows:
1. Catechin: 800 μL sodium citrate buffer (100 mM pH 5.5; final concentration 80 mM), 100 μL milliQ water and 100 μL catechin (10 mg/mL stock in DMSO; final concentration 1 mg/mL) were combined in a plastic Bijou pot and mixed gently.
2. Catechin/laccase: 700 μL sodium citrate buffer (100 mM pH 5.5), 100 μL milliQ water, 100 μL catechin (10 mg/mL stock in DMSO; final concentration 1 mg/mL) and 100 μL laccase (100 units/mL in citrate buffer pH 5.5) were combined in a plastic Bijou pot and mixed gently. The final concentration of sodium citrate buffer was 80 mM.

30 μL of each appropriate sample were then applied to the Vitro-Skin circles and rubbed in for 10 seconds using a gloved fingertip. Each sample was tested in triplicate. The Vitro-Skin was then incubated at 35° C. and 50% RH. CIE 1976 L*a*b* measurements for each sample area were recorded at various time-points up to 5 days and ΔE was calculated. Statistical analyses were performed using Student's t test.

Results

The results are set forth in Table 2a from which it is apparent that the colour change of (+)-catechin/laccase treated Vitro-Skin was significantly greater than the colour change of (+)-catechin treated Vitro-skin at all time points (p≧0.0002).

TABLE 2a
ΔE values for Vitro-Skin treated with (+)-catechin +/− laccase.
Standard deviations at 95% confidence limits are provided.

| | ΔE (+)- | ΔE (+)- | p value |
|---|---|---|---|
| | catechin- | catechin/laccase- | ΔE (+)-catechin vs ΔE (+)- |
| | treated | treated | catechin/laccase |
| 1 h | 0.74 +/− 0.71 | 7.11 +/− 0.08 | 0.0002 |
| 18 h | 0.87 +/− 0.37 | 6.48 +/− 0.31 | <0.0001 |
| 24 h | 1.28 +/− 0.43 | 6.71 +/− 0.36 | 0.0002 |
| 42 h | 0.63 +/− 0.27 | 7.26 +/− 0.47 | <0.0001 |
| 48 h | 0.95 +/− 0.53 | 7.21 +/− 0.42 | 0.0002 |
| 98 h | 1.33 +/− 0.35 | 7.20 +/− 0.47 | <0.0001 |

Table 2b shows the CIE 1976 L*a*b* (CIELAB) values at the 98 hour time-point from which it is clear that a noticeable browner colour was achieved after treatment with (+)-catechin/laccase compared to treatment with (+)-catechin alone.

TABLE 2b
L*a*b* values for untreated Vitro-Skin and Vitro-Skin treated with
(+)-catechin +/− laccase for 98 hours. Standard deviations at
95% confidence limits are provided.

| | | | (+)-catechin/laccase |
|---|---|---|---|
| | Untreated | (+)-catechin treated | treated |
| L* | 89.25 +/− 0.53 | 88.31 +/− 0.10 | 90.86 +/− 0.38 |
| a* | 1.29 +/− 0.02 | 0.84 +/− 0.14 | 1.41 +/− 0.09 |
| b* | −7.13 +/− 0.19 | −7.06 +/− 0.08 | −0.18 +/− 0.52 |

Conclusions

These results demonstrate that, on artificial skin, a significantly greater colour development is obtained after treatment with (+)-catechin and laccase, compared to the colour development obtained after treatment with (+)-catechin alone.

### Example 3

### A Comparison of the Colour Development on Artificial Skin Following Treatment with (−)-epicatchin, or (−)-epicatechin and Horseradish Peroxidase

Materials (Additional)

(−)-Epicatechin, Sigma, UK

Method

Vitro-Skin was prepared as described in Example 1.

Two samples were prepared as follows:
1. (−)-Epicatechin: 800 μL sodium citrate buffer (100 mM pH 5.5; final concentration 80 mM), 100 μL milliQ water and 100 μL (−)-epicatechin (10 mg/mL stock in DMSO; final concentration 1 mg/mL) were combined in a plastic Bijou pot and mixed gently.
2. (−)-Epicatechin/horseradish peroxidase: 700 μL sodium citrate buffer (100 mM pH 5.5), 100 μL 3% hydrogen peroxide (final concentration 0.3%), 100 μL (−)-epicatechin (10 mg/mL stock in DMSO; final concentration 1 mg/mL) and 100 μL horseradish peroxidase (100 units/mL in citrate buffer pH 5.5) were combined in a plastic Bijou pot and mixed gently. The final concentration of sodium citrate buffer was 80 mM.

30 μL of each appropriate sample were then applied to the Vitro-skin circles and rubbed in for 10 seconds using a gloved fingertip. Each sample was tested in triplicate. The Vitro-Skin was then incubated at 35° C. and 50% RH. CIE 1976 L*a*b* measurements for each sample area were recorded at various time-points up to 6 days and ΔE was calculated. Statistical analyses were performed using Student's t test.

Results

The results are set forth in Table 3a from which it is apparent that the colour change of (−)-epicatechin/horseradish peroxidase treated Vitro-Skin was significantly greater than the colour change of (−)-epicatechin treated Vitro-skin at all time points (p<0.0001).

TABLE 3a
ΔE values for Vitro-Skin treated with epicatechin +/− horseradish
peroxidase. Standard deviations at 95% confidence limits are provided.

| | | | p value |
|---|---|---|---|
| | ΔE (−)- | ΔE (−)- | ΔE |
| | epicatechin | epicatechin/HRP | (−)-epicatechin vs ΔE (−)- |
| | treated | treated | epicatechin/HRP |
| 1 h | 0.52 +/− 0.12 | 9.70 +/− 0.16 | <0.0001 |
| 23 h | 1.32 +/− 0.21 | 11.55 +/− 0.69 | <0.0001 |
| 28 h | 1.76 +/− 0.24 | 12.07 +/− 0.52 | <0.0001 |
| 47 h | 1.51 +/− 0.20 | 12.22 +/− 0.58 | <0.0001 |
| 52 h | 1.17 +/− 0.23 | 13.09 +/− 0.43 | <0.0001 |
| 72 h | 1.54 +/− 0.41 | 12.82 +/− 0.62 | <0.0001 |
| 122 h | 1.57 +/− 0.28 | 13.82 +/− 0.55 | <0.0001 |

Table 3b shows the CIE 1976 L*a*b* (CIELAB) values at the 122 hour time-point from which it is clear that a noticeable browner colour was achieved after treatment with (−)-epicatechin/horseradish peroxidase compared to treatment with (−)-epicatechin alone.

TABLE 3b
L*a*b* values for untreated Vitro-Skin and Vitro-Skin treated with
epicatechin +/− horseradish peroxidase for 122 hours. Standard deviations
at 95% confidence limits are provided.

| | | (−)-Epicatechin | (−)-Epicatechin/HRP |
|---|---|---|---|
| | Untreated | treated | treated |
| L* | 89.41 +/− 1.49 | 87.62 +/− 1.08 | 90.16 +/− 0.19 |
| a* | 1.27 +/− 0.18 | 1.29 +/− 0.06 | 3.40 +/− 0.17 |
| b* | −6.8 +/− 0.30 | −7.47 +/− 0.14 | 6.74 +/− 0.90 |

Conclusions

These results demonstrate that, on artificial skin, a significantly greater colour development is obtained after treatment with (−)-epicatechin and horseradish peroxidase, compared to the colour development obtained after treatment with (−)-epicatechin alone.

### Example 4

### A Comparison of the Colour Development on Artificial Skin Following Treatment with Green Tea Extract (Sunphenon), or Green Tea Extract (Sunphenon) and Horseradish Peroxidase

Materials (Additional)

Sunphenon 90 LB green tea extract, Taiyo, Japan

Sunphenon 90 LB, obtained from the leaf of traceable green tea (Camellia sinensis), is a series of highly purified polyphenols rich in natural green tea catechins. Sunphenon 90 LB contains 80% minimum total polyphenols, 80% minimum catechins, 40% minimum (−)-epigallocatechin gallate.

Method

Vitro-Skin was prepared as described in Example 1.

Two samples were prepared as follows:
1. Sunphenon 90 LB: 800 μL sodium citrate buffer (100 mM pH 5.5; final concentration 80 mM), 100 μL milliQ water and 100 μL Sunphenon 90 LB (100 mg/mL stock in DMSO; final concentration 10 mg/mL) were combined in a plastic Bijou pot and mixed gently.
2. Sunphenon 90 LB/horseradish peroxidase: 700 μL sodium citrate buffer (100 mM pH 5.5), 100 μL 3% hydrogen peroxide (final concentration 0.3%), 100 μL Sunphenon 90 LB (100 mg/mL stock in DMSO; final concentration 10 mg/mL) and 100 μL horseradish peroxidase (100 units/mL in citrate buffer pH 5.5) were combined in a plastic Bijou pot and mixed gently. The final concentration of sodium citrate buffer was 80 mM.

30 μL of each appropriate sample were then applied to the Vitro-Skin circles and rubbed in for 10 seconds using a gloved fingertip. Each sample was tested in triplicate. The Vitro-Skin was then incubated at 35° C. and 50% RH. CIE 1976 L*a*b* measurements for each sample area were recorded at various time-points up to 4 days and ΔE was calculated. Statistical analyses were performed using Student's t test.

Results

The results are set forth in Table 4a from which it is apparent that the colour change of Sunphenon 90 LB/horseradish peroxidase treated Vitro-Skin was significantly greater than the colour change of Sunphenon 90 LB treated Vitro-skin at all time points (p<0.0001).

TABLE 4a
ΔE values for Vitro-Skin treated with Sunphenon 90 LB green tea
extract +/− horseradish peroxidase. Standard deviations at 95%
confidence limits are provided.

| | | | p value |
|---|---|---|---|
| | ΔE | | ΔE Sunphenon |
| | Sunphenon 90 | ΔE Sunphenon 90 LB/ | 90 LB vs ΔE |
| | LB treated | HRP treated | Sunphenon 90 LB/HRP |
| 1 h | 0.60 +/− 0.15 | 4.97 +/− 0.37 | 0.0001 |
| 18 h | 0.73 +/− 0.08 | 5.28 +/− 0.19 | <0.0001 |
| 24 h | 1.39 +/− 0.31 | 6.22 +/− 0.12 | <0.0001 |
| 50 h | 3.35 +/− 0.33 | 7.46 +/− 0.06 | <0.0001 |
| 96 h | 5.38 +/− 0.17 | 9.23 +/− 0.07 | <0.0001 |

Table 4b shows the CIE 1976 L*a*b* (CIELAB) values at the 96 hour time-point from which it is clear that a noticeable browner colour was achieved after treatment with Sunphenon 90 LB/horseradish peroxidase compared to treatment with Sunphenon 90 LB alone.

TABLE 4b
L*a*b* values for untreated Vitro-Skin and Vitro-Skin treated with
Sunphenon 90 LB green tea extract +/− horseradish peroxidase for
96 hours. Standard deviations at 95% confidence limits are provided.

| | | Sunphenon 90 LB | Sunphenon 90 |
|---|---|---|---|
| | Untreated | treated | LB/HRP treated |
| L* | 86.90 +/− 0.04 | 87.54 +/− 0.51 | 85.95 +/− 0.38 |
| a* | 0.77 +/− 0.11 | 3.06 +/− 0.09 | 3.30 +/− 0.14 |
| b* | −5.67 +/− 0.06 | −0.94 +/− 0.05 | 3.14 +/− 0.15 |

Conclusions

These results demonstrate that, on artificial skin, a significantly greater colour development is obtained after treatment with Sunphenon 90 LB green tea extract and horseradish peroxidase, compared to the colour development obtained after treatment with Sunphenon 90 LB green tea extract alone.

### Example 5

### Colour Development on Artificial Skin Following Repeated Application of (+)-catechin and Horseradish Peroxidase

Method

Vitro-Skin was prepared as described in Example 1.

A (+)-catechin/horseradish peroxidase sample was prepared as follows: 400 μL sodium citrate buffer (100 mM, pH 5.5), 200 μL 3% hydrogen peroxide, 200 μL (+)-catechin (10 mg/mL stock in DMSO) and 200 μL horseradish peroxidase (100 units/mL in citrate buffer, pH 5.5) were combined in a plastic Bijou pot and mixed gently. 1 mL sodium citrate buffer (100 mM, pH 5.5) was then added to the combined mixture and the sample was mixed gently again. Final concentrations were as follows: sodium citrate buffer 80 mM; hydrogen peroxide 0.3%; (+)-catechin 1 mg/mL.

30 μL of the (+)-catechin/horseradish peroxidase sample were then applied to 6 Vitro-Skin circles and rubbed in for 10 seconds using a gloved fingertip. The Vitro-Skin was then incubated at 35° C. and 50% RH. At 24 hours, 48 hours, 72 hours, 98 hours and 121 hours after initial application, the (+)-catechin/horseradish peroxidase sample was reapplied to 3 Vitro-Skin circles. The sample was not re-applied to the other 3 Vitro-Skin circles. The CIE 1976 L*a*b* measurements for each sample area were recorded at 24 hours, 48 hours, 72 hours, 98 hours, 121 hours and 148 hours after the initial application (before re-applying sample) and ΔE was calculated. Statistical analyses were performed using Student's t test.

Results

The results are set forth in Table 5a from which it is apparent that repeated application of (+)-catechin and horseradish peroxidase on Vitro-Skin produces a significantly greater colour change (P<0.0001).

TABLE 5a
ΔE values for Vitro-Skin treated with (+)-catechin/horseradish
peroxidase. The effect of repeated application is shown at 24 hours,
48 hours, 72 hours, 98 hours and 121 hours after initial application.
Standard deviations at 95% confidence limits are provided.

| | | ΔE (+)- | p value |
|---|---|---|---|
| | | catechin/HRP | ΔE (+)- |
| | ΔE (+)- | treated | catechin/HRP vs ΔE (+)- |
| | catechin/HRP | repeated | catechin/HRP repeated |
| | treated | application | application |
| 1 h | 9.10 +/− 0.21 | 9.19 +/− 0.21 | 0.70 |
| 4 h | 5.98 +/− 0.30 | 6.10 +/− 0.35 | 0.75 |
| 24 h | 4.93 +/− 0.27 | 5.01 +/− 0.33 | 0.81 |
| 48 h | 5.16 +/− 0.15 | 9.72 +/− 0.32 | <0.0001 |
| 72 h | 5.63 +/− 0.19 | 14.11 +/− 0.07 | <0.0001 |
| 98 h | 5.88 +/− 0.31 | 18.24 +/− 0.15 | <0.0001 |
| 121 h | 6.15 +/− 0.40 | 21.90 +/− 0.56 | <0.0001 |
| 148 h | 6.29 +/− 0.17 | 23.19 +/− 0.40 | <0.0001 |

Table 5b shows the CIE 1976 L*a*b* (CIELAB) values at 24, 72 and 148 hour time-points from which it is clear that a noticeable browner colour was achieved after repeated treatment with (+)-catechin/horseradish peroxidase.

TABLE 5b
CIE 1976 L*a*b* values for untreated Vitro-Skin and Vitro-Skin
repeatedly treated with (+)-catechin/horseradish peroxidase.
Standard deviations at 95% confidence limits are provided.

| | | (+)-Catechin/HRP-treated | | |
|---|---|---|---|---|
| | | 24 h | | 148 h |
| | | after initial | 72 h after initial | after initial |
| | Untreated | treatment | treatment | treatment |
| L* | 88.78 +/− 0.91 | 89.09 +/− 0.31 | 88.12 +/− 0.07 | 86.96 +/− 0.10 |
| a* | 0.88 +/− 0.16 | 1.19 +/− 0.05 | 0.60 +/− 0.10 | 0.49 +/− 0.06 |
| b* | −4.96 +/− 0.05 | −0.08 +/− 0.30 | 9.10 +/− 0.04 | 18.13 +/− 0.32 |

Conclusions

These results demonstrate that, on artificial skin, a significantly greater colour development is obtained after repeated application of (+)-catechin and horseradish peroxidase, compared to the colour development obtained after a single application of (+)-catechin and horseradish peroxidase.

### Example 6

### Colour Development on Artificial Skin Following Treatment with (+)-catechin/horseradish Peroxidase in Combination with DHA and Selected Amino Acids

Materials (Additional)

1,3-Dihydroxyacetone dimer (DHA), Sigma, UK.

Glycerol, Sigma, UK

Sodium citrate buffer pH 5.6

Glycine, Sigma, UK

L-lysine, Sigma, UK

L-arginine, Sigma, UK

L-cysteine, Sigma, UK

Method

Vitro-Skin was prepared as described in Example 1.

A (+)-catechin/horseradish peroxidase stock solution was prepared as follows: 400 μL sodium citrate buffer (100 mM, pH 5.5), 200 μL 3% hydrogen peroxide, 200 μL catechin (10 mg/mL stock in DMSO) and 200 μL HRP (100 units/mL in citrate buffer, pH 5.5) were combined in a plastic Bijou pot and mixed gently.

To prepare the DHA/amino acid stock solutions 5 wt % DHA and 5 wt % amino acid were dissolved in glycerol/citrate buffer, pH 5.6 (0.15 glycerol/0.85 citrate buffer). The DHA/amino acids samples were then prepared by mixing 1 mL of the DHA/amino acid stock solution with 1 mL glycerol/citrate buffer (0.15/0.85). Final concentrations were as follows: 2.5 wt % DHA; and 2.5 wt % amino acid.

The DHA/amino acid/(+)-catechin/horseradish peroxidase samples were prepared by mixing 1 mL of the DHA/amino acid stock solution with 1 mL of the (+)-catechin/horseradish peroxidase stock solution. Final concentrations were as follows: 2.5 wt % DHA; 2.5 wt % amino acid; 0.3% hydrogen peroxide; and 1 mg/mL (+)-catechin.

30 μL of each appropriate sample were then applied to the Vitro-Skin circles and rubbed in for 10 seconds using a gloved fingertip. Each sample was tested in triplicate. The Vitro-Skin was then incubated at 35° C. and 50% RH. CIE 1976 L*a*b* measurements for each sample area were recorded 1 hour after sample application and ΔE was calculated. Statistical analyses were performed using Student's t test.

Results

The results in Table 6a show that the colour change of DHA/amino acid/(+)-catechin/horseradish peroxidase treated Vitro-Skin was significantly greater than the colour change of DHA/amino acid treated Vitro-Skin 1 hour after treatment for amino acids glycine, lysine, arginine and cysteine.

TABLE 6a
ΔE values for Vitro-Skin treated with DHA/amino acid +/− (+)-
catechin/horseradish peroxidase 1 hour after treatment.
Standard deviations at 95% confidence limits are provided.

| | | | p value |
|---|---|---|---|
| | | | ΔE DHA/amino |
| | | | acid vs |
| | | | ΔE DHA/amino |
| Amino | ΔE DHA/ | ΔE DHA/amino acid/ | acid/(+)-catechin/ |
| acid | amino acid | (+)-catechin/HRP | HRP |
| Glycine | 1.52 +/− 0.29 | 8.47 +/− 0.70 | 0.0002 |
| Lysine | 2.19 +/− 0.46 | 10.23 +/− 0.31 | <0.0001 |
| Arginine | 0.97 +/− 0.34 | 5.71 +/− 0.60 | 0.0006 |
| Cysteine | 0.79 +/− 0.19 | 11.42 +/− 0.20 | <0.0001 |

Tables 6b to 6e show the corresponding CIE 1976 L*a*b* (CIELAB) values for untreated Vitro-Skin and Vitro-Skin treated with DHA/amino acid +/−(+)-catechin/horseradish peroxidase for each of the amino acids glycine, lysine, arginine and cysteine respectively, from which it is clear that a noticeable browner colour was achieved after treatment with DHA/amino acid/(+)-catechin/horseradish peroxidase.

TABLE 6b
CIE 1976 L*a*b* values for untreated Vitro-Skin and Vitro-Skin
treated with DHA/glycine +/− (+)-catechin/horseradish peroxidase
for 1 hour. Standard deviations at 95% confidence limits are provided.

| | | | DHA/glycine/ |
|---|---|---|---|
| | | | (+)-catechin/HRP- |
| | Untreated | DHA/glycine-treated | treated |
| L* | 89.70 +/− 0.59 | 89.34 +/− 0.38 | 90.78 +/− 0.47 |
| a* | 0.58 +/− 0.07 | 0.72 +/− 0.04 | 1.20 +/− 0.02 |
| b* | −6.28 +/− 0.16 | −5.10 +/− 0.16 | 2.02 +/− 0.71 |

TABLE 6c
CIE 1976 L*a*b* values for untreated Vitro-Skin and Vitro-Skin
treated with DHA/lysine +/− (+)-catechin/horseradish peroxidase
for 1 hour. Standard deviations at 95% confidence limits are provided.

| | | | DHA/lysine/ |
|---|---|---|---|
| | | | (+)-catechin/HRP- |
| | Untreated | DHA/lysine-treated | treated |
| L* | 88.24 +/− 0.87 | 87.10 +/− 1.13 | 91.00 +/− 0.39 |
| a* | 0.84 +/− 0.13 | 0.89 +/− 0.07 | 0.55 +/− 0.09 |
| b* | −6.45 +/− 0.06 | −5.50 +/− 0.18 | 3.36 +/− 0.11 |

TABLE 6d
CIE 1976 L*a*b* values for untreated Vitro-Skin and Vitro-Skin
treated with DHA/arginine +/− (+)-catechin/horseradish peroxidase
for 1 hour. Standard deviations at 95% confidence limits are provided.

| | | | DHA/arginine/ |
|---|---|---|---|
| | | DHA/arginine- | (+)-catechin/HRP- |
| | Untreated | treated | treated |
| L* | 89.80 +/− 0.74 | 88.54 +/− 0.41 | 90.57 +/− 0.35 |
| a* | 0.83 +/− 0.14 | 0.93 +/− 0.03 | 0.92 +/− 0.08 |
| b* | −6.30 +/− 0.14 | −5.96 +/− 0.05 | −0.66 +/− 0.56 |

TABLE 6e
CIE 1976 L*a*b* values for untreated Vitro-Skin and Vitro-Skin
treated with DHA/cysteine +/− (+)-catechin/horseradish peroxidase
for 1 hour. Standard deviations at 95% confidence limits are provided.

| | | | DHA/cysteine/ |
|---|---|---|---|
| | | DHA/cysteine- | (+)-catechin/HRP- |
| | Untreated | treated | treated |
| L* | 88.39 +/− 0.87 | 89.00 +/− 0.16 | 90.25 +/− 0.50 |
| a* | 0.97 +/− 0.05 | 0.91 +/− 0.07 | 0.99 +/− 0.09 |
| b* | −6.57 +/− 0.12 | −6.47 +/− 0.16 | 4.63 +/− 0.23 |

Conclusions

These results demonstrate that, for the amino acids glycine, lysine, arginine and cysteine, a significantly greater colour development is obtained on artificial skin one hour after treatment with 1,3-dihydroxyacetone dimer/amino acid/(+)-catechin/horseradish peroxidase, compared to the colour development obtained on artificial skin one hour after treatment with 1,3-dihydroxyacetone dimer/amino acid.

### Example 7

### A comparison of Colour Development on Artificial Skin Following Treatment by DHA/arginine or DHA/cysteine Compared to Treatment by DHA/arginine/(+)-catechin/horseradish Peroxidase or DHA/cysteine/(+)-catechin/horseradish Peroxidase

Method

Vitro-Skin was prepared as described in Example 1.

A (+)-catechin/horseradish peroxidase stock solution, DHA/arginine stock solution and a DHA/cysteine stock solution were prepared as described in Example 6.

The DHA/amino acid samples were then prepared by mixing 1 mL of the DHA/amino acid stock solution with 1 mL glycerol/citrate buffer (0.15/0.85). Final concentrations were as follows: 2.5 wt % DHA; and 2.5 wt % amino acid.

The DHA/amino acid/(+)-catechin/horseradish peroxidase samples were prepared by mixing 1 mL of the DHA/amino acid stock solution with 1 mL of the catechin/horseradish peroxidase stock solution. Final concentrations were as follows: 2.5 wt % DHA; 2.5 wt % amino acid; 0.3% hydrogen peroxide; and 1 mg/mL (+)-catechin.

30 μL of each appropriate sample were then applied to the Vitro-Skin circles and rubbed in for 10 seconds using a gloved fingertip. Each sample was tested in triplicate. The Vitro-Skin was then incubated at 35° C. and 50% RH. CIE 1976 L*a*b* measurements for each sample area were recorded at various time-points up to 5 days and ΔE was calculated. Statistical analyses were performed using Student's t test.

Results

The results in Tables 7a and 7b show that the colour changes of DHA/arginine/(+)-catechin/horseradish peroxidase or DHA/cysteine/(+)-catechin/horseradish peroxidase treated Vitro-Skin were significantly greater than the colour change of DHA/arginine or DHA/cysteine treated Vitro-Skin at all time points (p=<0.05).

TABLE 7a
ΔE values for Vitro-Skin treated with DHA/arginine +/− (+)-catechin/
horseradish peroxidase. Standard deviations at 95% confidence limits are
provided.

| | | | p value |
|---|---|---|---|
| | ΔE DHA/ | | ΔE DHAarginine/ |
| | arginine | ΔE DHA/arginine/(+)- | vs ΔE DHA/arginine/ |
| | treated | catechin/HRP treated | (+)-catechin/HRP |
| 1 h | 0.97 +/− 0.34 | 5.71 +/− 0.60 | 0.0006 |
| 23 h | 14.04 +/− 0.11 | 15.90 +/− 0.39 | 0.0028 |
| 48 h | 16.26 +/− 0.29 | 17.83 +/− 0.67 | 0.038 |
| 71 h | 18.42 +/− 0.19 | 19.87 +/− 0.70 | 0.047 |

TABLE 7b
ΔE values for Vitro-Skin treated with DHA/cysteine +/− (+)-catechin/
horseradish peroxidase. Standard deviations at 95% confidence limits are
provided.

| | | | p value ΔE |
|---|---|---|---|
| | ΔE DHA/ | | DHA/cysteine vs ΔE |
| | cysteine | ΔE DHA/cysteine/(+)- | DHA/cysteine/(+)- |
| | treated | catechin/HRP treated | catechin/HRP |
| 1 h | 0.79 +/− 0.19 | 11.42 +/− 0.20 | <0.0001 |
| 23 h | 8.11 +/− 0.12 | 19.89 +/− 0.26 | <0.0001 |
| 48 h | 13.99 +/− 0.45 | 22.88 +/− 0.35 | <0.0001 |
| 71 h | 17.19 +/− 0.66 | 25.57 +/− 0.40 | 0.0001 |
| 120 h | 18.61 +/− 0.90 | 26.24 +/− 0.71 | 0.0007 |

Tables 7c and 7d show the corresponding CIE 1976 L*a*b* (CIELAB) values for untreated Vitro-Skin and Vitro-Skin treated with DHA/amino acid +/−(+)-catechin/horseradish peroxidase for the amino acids arginine 71 hours after treatment and cysteine 120 hours after treatment respectively, from which it is clear that a noticeable browner colour was achieved after treatment with DHA/amino acid/(+)-catechin/horseradish peroxidase.

TABLE 7c
L*a*b* values for untreated Vitro-Skin and Vitro-Skin treated with
DHA/arginine +/− catechin/HRP for 71 hours. Standard deviations
at 95% confidence limits are provided.

| | | DHA/arginine | DHA/arginine/(+)- |
|---|---|---|---|
| | Untreated | treated | catechin/HRP treated |
| L* | 89.80 +/− 0.74 | 88.52 +/− 0.18 | 88.86 +/− 0.57 |
| a* | 0.83 +/− 0.14 | 1.62 +/− 0.08 | 1.29 +/− 0.14 |
| b* | −6.30 +/− 0.14 | 11.93 +/− 0.14 | 13.51 +/− 0.51 |

TABLE 7d
L*a*b* values for untreated Vitro-Skin and Vitro-Skin treated with
DHA/cysteine +/− catechin/HRP for 120 hours.
Standard deviations at 95% confidence limits are provided.

| | | DHA/cysteine | DHA/cysteine/(+)- |
|---|---|---|---|
| | Untreated | treated | catechin/HRP treated |
| L* | 88.39 +/− 0.87 | 89.10 +/− 0.43 | 85.69 +/− 0.90 |
| a* | 0.97 +/− 0.05 | 1.05 +/− 0.13 | 2.92 +/− 0.14 |
| b* | −6.57 +/− 0.12 | 11.94 +/− 0.98 | 19.42 +/− 0.50 |

Conclusions

These results demonstrate that, for the amino acids arginine and cysteine, a significantly greater colour development is obtained on artificial skin up to 71 and 120 hours respectively after treatment with 1,3-dihydroxyacetone (DHA)/amino acid/(+)-catechin/horseradish peroxidase, compared to the colour development obtained on artificial skin after treatment with 1,3-dihydroxyacetone (DHA)/amino acid.

### Example 8

### A Comparison of the Colour Development on Bleached Human Hair Following Treatment with Apigenin with and Without Horseradish Peroxidase

Materials (Additional)

Human natural white hair switches (International Hair Importers, USA)

Platine precision lightening powder (50% persulphates, 24.1% silicates and 2.6% ammonium chloride) (L'Oreal, France)

Excel cream peroxide, 9%. (Excel (GS) Ltd., UK)

Apigenin (Sigma, UK)

Britton-Robinson buffer

Methods

Hair Bleaching

2″ natural white hair switches were bleached twice using L'Oreal Platine Precision lightening powder and Excel cream peroxide according to the manufacturers' instructions (30 minutes each treatment).

Hair Treatment

4200 μL Britton-Robinson buffer (62.5 mM pH 5; final concentration 50 mM)

600 μL hydrogen peroxide 3%

600 μL apigenin, 10mg/mL stock in DMSO

Prior to treatment, the colour (L*a*b*) of each switch was recorded using a Minolta CM-2600d spectrophotometer. The reagents listed above were then combined in 15 mL Falcon centrifuge tubes. Bleached hair switches were placed into the tubes and squashed down, wetting the hair thoroughly (one switch per tube). The switch/reagents were then incubated at 32° C. for 15 minutes. 30 Units horseradish peroxidase (HRP VI, in Britton-Robinson buffer pH 5, total volume 600 μL) or 600 μL Britton-Robinson buffer pH 5 were then added as appropriate to the tubes. The hair switches were then incubated at 32° C. for another 15 minutes. After incubation the switches were washed by rinsing under running tap water for 1 minute. The hair was then shampoo washed by rubbing with shampoo for 30 seconds and subsequently rinsing under tap water for 60 seconds. The hair was then dried with a hairdryer and combed. The above method was repeated twice (3 treatments in total). After each treatment the colour of each switch (L*a*b*) was recorded and AE was calculated. Untreated switches were washed 3 times with shampoo, rinsed and dried as described above.

Results

After addition of the horseradish peroxidase, the solution was observed to have an off-white colour. The ΔE values for each switch after three treatments are shown in Table 8. A ΔE value of 5 or above was considered to colour hair. Accordingly, treatment with apigenin with or without horseradish peroxidase did not colour hair.

TABLE 8
ΔE values for double bleached natural white hair treated three
times with apigenin with and without horseradish peroxidase.

| | ΔE |
|---|---|
| No treatment | 1.10 |
| Apigenin | 2.13 |
| Apigenin/horseradish peroxidase | 1.72 |

Conclusions

Treatment of double bleached natural white hair with apigenin with or without horseradish peroxidase did not colour hair. Therefore it can be expected that, particularly as the solution after addition of horseradish peroxidase was observed to be white, apigenin with or without horseradish peroxidase will not colour skin, which is a structurally related organ to hair.

### Example 9

### A Comparison of the Colour Development on Unbleached and Bleached Human Hair Following Treatment with Chlorogenic Acid with and Without Horseradish Peroxidase

Materials (Additional)

Chlorogenic acid (Sigma, UK)

Horseradish peroxidase, 53 U/mg (1 Unit=1 mg purpurogallin in 20 s at 20° C. and pH 6) (HRP I, Sigma, UK)

Methods

Hair Bleaching

If required, natural white hair switches were bleached twice using L'Oreal Platine Precision lightening powder and Excel cream peroxide according to the manufacturers' instructions (30 minutes each treatment).

Hair Treatment

Prior to treatment, the colour (L*a*b*) of each switch was recorded using a Minolta CM-2600d spectrophotometer. 700 μL Britton-Robinson buffer (62.5 mM pH 6, final concentration 50 mM), 100 μL 3% H₂O₂ (horseradish peroxidase treated switches) or 100 μL milliQ water (non horseradish peroxidase treated switches), and 100 μL chlorogenic acid (100 mg/mL stock in DMSO) were combined in plastic Bijou pots. 2″ unbleached and bleached natural white switches were then placed into the pots and squashed down (one switch per pot), wetting the hair thoroughly. The switch/reagents were then incubated at 37° C. for 5 minutes. 100 μL horseradish peroxidase (HRP I, 1 mg/mL in BR buffer, pH6) (horseradish peroxidase treated switches) or 100 μL Britton-Robinson buffer (non horseradish peroxidase treated switches) were then added and rubbed into the switch. Each hair switch was then placed back into the pot and incubated at 37° C. for 5 minutes. After 5 minutes incubation the switch was washed by swirling in a beaker of MilliQ water for approx. 2 minutes. The hair was dried with a hairdryer and combed through. The above method was repeated twice (3 treatments in total). After each treatment the colour of each switch (L*a*b*) was recorded and ΔE was calculated.

Results

After addition of horseradish peroxidase the solution was observed to have a brown colour. The ΔE values for each switch after three treatments are shown in Table 9. A ΔE value of 5 or above was considered to colour hair. Treatment with chlorogenic acid with or without horseradish peroxidase did not colour hair, whether bleached or unbleached.

TABLE 9
ΔE values for unbleached and double bleached natural white hair treated
three times with chlorogenic acid with and without horseradish peroxidase.

| | Unbleached | Bleached |
|---|---|---|
| | hair | hair |
| | ΔE | ΔE |
| Chlorogenic acid | 1.64 | 1.23 |
| Chlorogenic acid/horseradish | 1.71 | 4.18 |
| peroxidase | | |

Conclusions

Treatment of unbleached or double bleached natural white hair with chlorogenic acid with or without horseradish peroxidase did not colour hair. Therefore it can be expected that chlorogenic acid with or without horseradish peroxidase will not colour skin, which is a structurally related organ to hair.

### Example 10

### A Comparison of the Colour Development on Unbleached and Bleached Human Hair Following Treatment with (+)-catechin with or without Horseradish Peroxidase

Methods

Hair Bleaching

If required, natural white hair switches were bleached twice using L'Oreal Platine Precision lightening powder and Excel cream peroxide according to the manufacturers' instructions (30 minutes each treatment).

Hair Treatment

Prior to treatment, the colour (L*a*b*) of each switch was recorded using a Minolta CM-2600d spectrophotometer. 700 μL Britton-Robinson buffer (62.5 mM pH 6, final concentration 50 mM), 100 μL 3% H₂O₂ (horseradish peroxidase treated switches) or 100 μL milliQ water (non horseradish peroxidase treated switches), and 100 μL (+)-catechin (100 mg/mL stock in DMSO) were combined in plastic Bijou pots. 2″ unbleached and bleached natural white switches were then placed into the pots and squashed down (one switch per pot), wetting the hair thoroughly. The switch/reagents were then incubated at 37° C. for 5 minutes. 100 μL horseradish peroxidase (HRP I, 1 mg/mL in BR buffer, pH6) (horseradish peroxidase treated switches) or 100 μL Britton-Robinson buffer (non horseradish peroxidase treated switches) were then added and rubbed into the switch. Each hair switch was then placed back into the pot and incubated at 37° C. for 5 minutes. After 5 minutes incubation the switch was washed by swirling in a beaker of MilliQ water for approx. 2 minutes. The hair was dried with a hairdryer and combed through. The above method was repeated twice (3 treatments in total). After each treatment the colour of each switch (L*a*b*) was recorded and ΔE was calculated.

Results

After addition of horseradish peroxidase the solution was observed to have an orange brown colour. The ΔE values for each switch after one, two and three treatments are shown in Table 10. A ΔE value of 5 or above is considered to colour hair. Treatment with (+)-catechin without horseradish peroxidase did not colour hair, bleached or unbleached. Treatment with (+)-catechin/horseradish peroxidase coloured both bleached and unbleached hair.

TABLE 10
ΔE values for unbleached and double bleached natural white hair
treated once, twice and three times with (+)-catechin with and without
horseradish peroxidase.

| | Unbleached hair | Bleached hair |
|---|---|---|
| | ΔE | ΔE |
| (+)-catechin | | |
| Treatment 1 | 1.41 | 1.35 |
| Treatment 2 | 1.43 | 0.96 |
| Treatment 3 | 1.68 | 1.22 |
| (+)-catechin/horseradish peroxidase | | |
| Treatment 1 | 8.64 | 19.90 |
| Treatment 2 | 12.43 | 23.64 |
| Treatment 3 | 13.49 | 25.12 |

Conclusions

Treatment of unbleached or double bleached natural white hair with (+)-catechin without horseradish peroxidase did not colour hair. However treatment of unbleached or double bleached natural white hair with (+)-catechin/horseradish peroxidase coloured hair. This conclusion replicates the results observed on skin in Example 1.

### Example 11

### Colour Development on Bleached Human Hair Following Treatment with (−)-epicatechin and Horseradish Peroxidase, or (−)-epigallocatechin Gallate and Horseradish Peroxidase

Materials (Additional)

(−)-Epigallocatechin gallate, Sigma, UK

Methods

Hair Bleaching

2″ natural white hair switches were bleached twice using L'Oreal Platine Precision lightening powder and Excel cream peroxide according to the manufacturers' instructions (30 minutes each treatment).

Hair Treatment

4200 μL Britton-Robinson buffer (62.5 mM pH 5; final concentration 50 mM)

600 μL hydrogen peroxide 3%

600 μL (−)-epicatechin or (−)-epigallocatechin gallate, 10 mg/mL stock in DMSO

Prior to treatment, the colour (L*a*b*) of the switches was recorded using a Minolta CM-2600d spectrophotometer. The reagents listed above were then combined in 15 mL Falcon centrifuge tubes. Bleached hair switches were placed into the tubes and squashed down, wetting the hair thoroughly (one switch per tube). The switch/reagents were then incubated at 32° C. for 15 minutes. 30 Units horseradish peroxidase (HRP VI, in Britton-Robinson buffer pH 5, total volume 600 μL) were then added to each tube. The hair switches were then incubated at 32° C. for another 15 minutes. After incubation the switches were washed by rinsing under running tap water for 1 minute. The hair was then shampoo washed by rubbing with shampoo for 30 seconds and subsequently rinsing under tap water for 60 seconds. The hair was then dried with a hairdryer and combed. The above method was repeated twice (3 treatments in total). After each treatment the colour of each switch (L*a*b*) was recorded and AE was calculated.

Results

The ΔE values for each hair switch after one, two and three treatments are shown in Table 11. A ΔE value of 5 or above is considered to colour hair. Treatment of double bleached hair with (−)-epicatechin/horseradish peroxidase coloured hair. Treatment with (−)-epigallocatechin gallate/horseradish peroxidase coloured hair.

TABLE 11
ΔE values for double bleached natural white hair treated once,
twice and three times with (−)-epicatechin/horseradish peroxidase
or (−)-epigallocatechin gallate/horseradish peroxidase.

| | | (−)- |
|---|---|---|
| | (−)-Epicatechin/ | Epigallocatechin |
| | HRP ΔE | gallate/HRP ΔE |
| Treatment 1 | 15.43 | 5.75 |
| Treatment 2 | 19.25 | 9.86 |
| Treatment 3 | 24.07 | 12.21 |

Conclusions

Treatment of double bleached natural white hair with (−)-epicatechin/horseradish peroxidase coloured hair. Treatment of double bleached natural white hair with (−)-epigallocatechin gallate/horseradish peroxidase also coloured hair. These conclusions support the results seen for sunless tanning of artificial skin for (−)-epicatechin and (−)-epigallocatechin gallate observed in Examples 3 and 4 respectively.

1. A topical sunless tanning composition comprising:
(a) 0.01-10, preferably 0.1-5% w/w a polyphenol selected from the group consisting of (+)-catechin, (−)-catechin, (+)-epicatechin, (−)-epicatechin, (−)-epigallocatechin gallate, (−)-epigallocatechin, (−)-catechin gallate, epicatechin gallate, or mixtures thereof;
(b) A peroxidase or laccase
(c) 0.0001-3 preferably 0.001-1, most preferably 0.01-1% w/w hydrogen peroxide or a hydrogen peroxide generator when peroxidase is present;
(d) A dermatologically acceptable vehicle; and
(e) fragrance.

2. A topical sunless tanning composition according to claim 1, wherein the polyphenol is selected from the group consisting of (+)-catechin or (−)-epicatechin and (−)-epigallocatechin gallate.

3. A topical sunless tanning composition according to claim 1, wherein the peroxidase is a non-animal haem peroxidase from class II (fungi) or class Ill (plants and algae).

4. A topical sunless tanning composition according to claim 3, wherein the peroxidase is obtained from the group consisting of Arabidopsis thaliana, horse radish, barley, peanut soy bean, tobacco, and turnip (plants), Chlorophyta spirogyra (green algae), Arthromyces ramosus and Corprinus cinereus (fungi).

5. A topical sunless tanning composition according to claim 4 wherein the peroxidase is horse radish peroxidase or soy bean peroxidase.

6. A topical sunless tanning composition according to claim 1 wherein the laccase is selected from the group consisting of cyanobacteria of the genuses Leptolyngbya, Oscillatoria and Phormidium; bacteria of the genuses Bacillus, Escherichia, Pseudomonas, Shigella, Sinorhizobium, Stenotrophomonas, Streptomyces, and Thermus; fungi of the genuses Agaricus, Agrocybe, Albatrellus, Athelia, Botryotinia, Cantharellus, Ceriporiopsis, Cerrena, Chaetomium, Cladosporium, Clitocybe, Coniothyrium, Coprinopsis, Coriolisimus, Coriolopsis, Cortinarius, Cryptococcus, Cyathus, Daedalea, Emericella, Fomes, Fomitella, Fusarium, Ganoderma, Hypocrea, Inocybe, Lactarius, Lentinula, Lepiota, Lepista, Leptonia, Loweporus, Lyophyllum, Magnaporthe, Marasmius, Melanocarpus, Myceliophthora, Myriogonium, Myrothecium, Neurospora, Panus, Paraconiothyrium, Parasola, Peltigera, Penicillium, Peniophora, Perenniporia, Phellinus, Phlebia, Phoma, Pleurotus, Podospora, Polyporus, Pycnoporus, Ramaria, Rhizoctonia, Rigidoporus, Russula, Scytalidium, Solorina, Steccherinum, Trametes, Tricholoma, Trichophyton, Volvariella; plants of the genuses Acer, Mangifera, Pistacia, Pleiogynium, Populus, Prunus, Rhus, Schinus and Toxicodendron; and mixtures thereof.

7. A topical sunless tanning composition according to claim 1 wherein the hydrogen peroxide generator comprises a hydrogen peroxide generating oxidase, a substrate and oxygen.

8. A topical sunless tanning composition according to claim 7, wherein the hydrogen peroxide generating oxidase is selected from the group consisting of (S)-2-hydroxy acid oxidase, D-galactose oxidase, glucose oxidase, coniferyl alcohol oxidase, glycolate oxidase, hexose oxidase, oxalate oxidase, amino acid oxidase and L-galactonolactone oxidase and the respective substrate is selected from the group consisting of (S)-2-hydroxy acid, D-galactose, glucose, coniferyl alcohol, a-hydroxy acids, D-glucose, oxalic acid, amino acid and L-galactono-1,4-lactone.

9. A topical sunless tanning composition according to claim 1 wherein the hydrogen peroxide generator is selected from the group consisting of (S)-2-hydroxy acid with (S)-2-hydroxy acid oxidase, D-galactose with D-galactose oxidase, glucose with glucose oxidase, coniferyl alcohol with coniferyl alcohol oxidase, α-hydroxy acids with glycolate oxidase, D-glucose with hexose oxidase, oxalic acid with oxalate oxidase, and L-galactono-1,4-lactone with L-galactonolactone oxidase, amino acid oxidase with amino acids, all in the presence of oxygen.

10. A topical sunless tanning composition according to claim 1 further comprising 0.01-25, preferably 0.1-15, most preferably 0.1-10% w/w 1,3-dihydroxyacetone dimer and/or D-erythrulose.

11. A topical sunless tanning composition according to claim 10 further comprising 0.01-10, preferably 0.1-5% w/w an amino acid, preferably an amino acid excluding a thiol group.

12. A topical sunless tanning composition according to claim 11 wherein the amino acid is selected from the group consisting of glycine, L-lysine, L-arginine, L-cysteine and mixtures thereof, preferably selected from the group consisting of glycine, L-lysine, L-arginine and mixtures thereof.

13. A topical sunless tanning composition according to claim 1 further comprising 0.01 to 15, preferably 0.1 to 10, most preferably 0.5 to 7.5% w/w an inorganic sunscreen and/or organic sunscreen.

14. A method of sunless tanning the skin comprising the step of applying to the skin the topical sunless tanning composition of claim 1.
